(19) United States
(12) Patent Application Publication
(10) Pub. No.: US 2018/0070868 A1
(43) Pub. Date: Mar. 15, 2018

(54) APPARATUS FOR DETECTING AN ANALYTE AND METHOD OF OPERATING AND FORMING THE SAME

(71) Applicant: The Texas A&M University System, College Station, TX (US)

(72) Inventor: Pao T. Lin, College Station, TX (US)

(73) Assignee: The Texas A&M University System, College Station, TX (US)

(21) Appl. No.: 15/702,114

(22) Filed: Sep. 12, 2017

Related U.S. Application Data
(60) Provisional application No. 62/393,994, filed on Sep. 13, 2016.

(51) Int. Cl. A61B 5/1455; G02B 6/122; G01J 3/28; A61B 5/145

(52) U.S. Cl. CPC A61B 5/1455; G02B 6/122; G02B 2006/12097; A61B 5/14532; G01J 3/2823

(57) ABSTRACT

An apparatus for detecting an analyte, and method of operating and forming the same. In one embodiment, the apparatus includes a pedestal formed on a semiconductor substrate and a mid-infrared (“IR”) transparent semiconductor waveguide formed on the pedestal. A refractive index of the pedestal is less than the mid-IR transparent semiconductor waveguide. The apparatus also includes a detector configured to detect an analyte couplable to the mid-IR transparent semiconductor waveguide.

This application claims the benefit of U.S. Provisional Application No. 62/393,994 entitled “Apparatus for Detecting an Analyte and Method of Operating and Forming the Same,” filed Sep. 13, 2016, which is incorporated herein by reference.

### TECHNICAL FIELD

The present invention relates to an apparatus for detecting an analyte, and method of operating and forming the same.

### BACKGROUND

Diabetes is a chronic disease caused by elevated blood glucose levels that reach abnormal levels due to an insufficient production of insulin. Diabetes can also be caused by body cells that are unable to use insulin effectively. To control and to be able to treat diabetes, it is necessary to constantly monitor levels of the blood sugar and other important carbohydrates. Present glucose measurements like an enzymatic method employ specific labeling reagents such as glucose oxidase or hexokinase to differentiate glucose from other blood compounds. Additionally, for constant measurement, enzymes need to be stable over time, and that prevents the application to continuously trace glucose levels. Thus, an apparatus that effectively detects an analyte such as glucose, fructose, sucrose and lactose would be beneficial.

### SUMMARY

These and other problems are generally solved or circumvented, and technical advantages are generally achieved, by advantageous embodiments of the present invention, including an apparatus for detecting an analyte, and method of operating and forming the same. In one embodiment, the apparatus includes a pedestal formed on a semiconductor substrate and a mid-infrared (“IR”) transparent semiconductor waveguide formed on the pedestal. A refractive index of the pedestal is less than the mid-IR transparent semiconductor waveguide. The apparatus also includes a detector configured to detect an analyte couplable to the mid-IR transparent semiconductor waveguide.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, reference is now made to the following descriptions taken in conjunction with the accompanying drawings, in which:

FIG. 1 illustrates a graphical representation of exemplary properties of a nitride film;

FIGS. 2 and 3 illustrate diagrams of embodiments at least a portion of a sensor;

FIG. 4 illustrates a three-dimensional view of an embodiment of at least a portion of a sensor;

FIG. 5 illustrates views of an embodiment of at least a portion of a sensor;

FIG. 6 illustrates a graphical representation showing an exemplary refractive index profile of a portion of a sensor;

FIGS. 7 and 8 illustrate graphical representations showing exemplary field intensity profiles of a portion of a sensor;

FIG. 9 illustrates a graphical representation showing relative sensitivities as a function of a notch width of a pedestal of a sensor;

FIG. 10 illustrates mode images of different waveguides without and with alkyl carbohydrate;

FIG. 11 illustrates a graphical representation showing relative transmittance of glucose molecules measured by a waveguide;

FIG. 12 illustrates mode images of an aqueous glucose concentration;

FIG. 13 illustrates a graphical representation of relative waveguide mode intensity versus aqueous glucose concentration;

FIG. 14 illustrates a perspective view of an embodiment of a wearable sensor;

FIG. 15 illustrates a rear view of an embodiment of a retaining device for the apparatus for detecting an analyte of FIG. 14; and

FIG. 16 illustrates a flow diagram of an embodiment of a method for detecting an analyte.

Corresponding numerals and symbols in the different figures generally refer to corresponding parts unless otherwise indicated. The figures are drawn to clearly illustrate the relevant aspects of the preferred embodiments and are not necessarily drawn to scale.

### DETAILED DESCRIPTION OF ILLUSTRATIVE EMBODIMENTS

The making and using of the presently preferred embodiments are discussed in detail below. It should be appreciated, however, that the embodiments provide many applicable inventive concepts that can be embodied in a wide variety of specific contexts. The specific embodiments discussed are merely illustrative of specific ways to make and use the invention, and do not limit the scope of the invention.

Embodiments will be described in a specific context, namely, an apparatus including a waveguide (e.g., a mid-infrared (“IR”) transparent semiconductor waveguide) formed on a pedestal (e.g., a low refractive index bilayer pedestal) for detecting an analyte, and method of operating and forming the same. While the principles of the present invention will be described in the environment of detecting an analyte such as glucose, fructose, sucrose and lactose, any application or related technology that may benefit from an apparatus including a waveguide formed on a pedestal that can detect or otherwise characterize analyte is well within the broad scope of the present invention.

Mid-IR spectroscopy can perform real-time and label-free glucose detection. This is due to the strong characteristic photonic absorption of the glucose molecule in the mid-IR spectral region attributed to alkyl and hydroxyl functional groups. Thus, the body's glucose concentration can be accurately determined through mid-IR analysis using body fluidics such as serum or even epidermis. Using mid-IR quantum cascade lasers and mid-IR fibers, label-free glucose detections can be demonstrated. More recently, in vivo and noninvasive glucose measurements have been illustrated via tunable mid-IR lasers or via engineered hollow-core mid-IR fibers. Evidently, the mid-IR technology has revealed a potential to provide accurate and continuous glucose monitoring for a large population of diabetic patients.

Though mid-IR analysis is capable of performing label-free and real-time glucose measurements, present mid-IR spectroscopy employs bench-top optical equipment like Fourier transform infrared spectroscopy (“FTIR”) or a scanning monochromator that are bulky and are only practical for in-clinic or limited off-site glucose examinations. Current designs generally lead to bulky monitors that are not easily adaptable for wearable and continuous glucose monitoring, which are critical to maintaining proper diabetes control.

As introduced herein, a label-free sensor is formed with pedestal waveguides using a complementary metal-oxide semiconductor (“CMOS”) process. By using a CMOS process, high-volume manufacture of a sensor can be implemented to reduce fabrication cost, thereby providing a convenient and accurate testing device for a large population of diabetic patients or other applications. The device can be used, for example, in-house, outdoors, or in clinics. It can be miniaturized and integrated with wired and wireless devices for real-time monitoring.

Silicon nitride and aluminum nitride generally occur as molecules with the formulas SiₓN_{y}, AlₓN_{y}, respectively. However, it is recognized that other proportions of silicon or aluminum and nitrogen can occur in a silicon or aluminum nitride molecular structure. Accordingly, the compound silicon or aluminum nitride and its variants will be represented herein with the formulas SiN, AlN, respectively, which can also represent a solid solution of plural silicon or aluminum nitride molecular structures.

In contrast with current monitoring devices, the label-free sensor introduced herein does not employ a specific and often delicate labeling reagent such as glucose oxidase or hexokinase to differentiate glucose from other chemical compounds. By using integrated photonic components such as waveguides, a much higher glucose sensitivity can be achieved compared to present optical sensors because it has a much longer light-analyte interaction length. From Beer's law, a device with a longer sample path has a higher sensitivity. Sample path lengths of 1 centimeter (“cm”) or more can be achieved. On the other hand, a sensor using a conventional “microscope” geometry has a sample path length that is often less than 1 micrometer (“μm”).

The sensor as introduced herein can provide real-time glucose detection because no sample post-treatment is necessary. The sensor can perform continuous glucose detection without the need of a chemical replacement. Labeling reagents such as glucose oxidase or hexokinase are not consumed.

A glucose molecule has two major characteristic absorption bands at wavelengths between 2 μm and 6 μm, one at wavelengths from 2.73 to 3.10 μm from the hydroxyl (“—OH”) stretch of the glucose molecule, and the other one at wavelengths from 3.30 to 3.60 μm from the alkyl (“C—H”) stretch of the glucose molecule. To evaluate performance of the glucose sensors, spectral response within those two absorption bands can be recorded and analyzed. For aqueous glucose samples, mid-IR absorption at wavelengths greater than 3.55 μm is utilized for concentration measurements to prevent interference caused by water absorption. Water has a much lower photonic absorption compared to the glucose molecule for wavelengths greater than 3.55 μm. Meanwhile, a nitride (such as silicon or aluminum nitride) thin film may be employed for the sensor platform because its transparency window covers a broad mid-IR spectrum (wavelengths up to 8.5 μm), so the optical loss in the mid-IR waveguide is reduced. Additionally, to improve sensitivity, the waveguide design adopts a pedestal structure that has more sensing surface area compared to conventional ridge or rib waveguides. The waveguide mode profile and the sensitivity enhancement can be investigated by two-dimensional finite difference method (“FDM”) calculations. Given the advantage of label-free detection and scaling the device down to chip size, the mid-IR sensors can potentially be implanted into diabetes patients to enable real-time and continuous glucose monitoring.

To fabricate the nitride pedestal waveguide such as a silicon nitride pedestal waveguide, a low-stress 2 μm mid-IR transparent nitride (such as silicon nitride) thin film is deposited on a five μm low refractive index thermal oxide layer formed on a silicon (“Si”) substrate using a low-pressure chemical vapor deposition (“LPCVD”). In an embodiment, the silicon precursor is dichlorosilane and the nitrogen source is ammonia. The deposition pressure is set at 200 milliTorr (“mTorr”), and the temperature to 825 degrees Celsius (“° C.”), where a growth rate of 10 nanometers/minute (“nm/min”) is obtained. Before loading the silicon substrates into the LPCVD furnace, organic residues on silicon wafers are removed by a Piranha clean (3:1 volume ratio of sulfuric acid to 30 percent hydrogen peroxide).

The mid-IR pedestal waveguides may be fabricated on a silicon nitride thin film using photolithography. First, the desired waveguide structures are defined by a photo-mask through ultraviolet (“UV”) patterning. These layouts are then transferred into the silicon nitride thin film layer through inductively coupled plasma reactive ion etching (“ICP-RIE”) where, in an embodiment, the etch gases are argon (“Ar”), hydrogen (“H₂”), fluoroform (“CHF₃”) and carbon tetrafluoride (“CF₄”) with flow rates of 6, 30, 50 and 2 standard cubic centimeters per minute (“sccm”), respectively. Hexamethyldisilazane (“HMDS”) and a micron thick photoresist (Shipley 1813) are initially coated on the silicon nitride film with a speed of 4000 revolutions per minute (“rpm”), and the coated wafer is baked at 115° C. for one minute. Desired layouts including waveguides, splitters, and couplers are defined by a photomask through ultraviolet patterning, and are then developed using a Shipley MICROPOSIT™ MF-319 solution. These structures are transferred into the silicon nitride layer through an ICP-RIE etching process of 15 minutes in an Ar/H₂/CHF₃/CF₄ mixture with flow rates of 6/30/50/2 sccm, respectively. The silicon nitride etching rate is 150 nm/min, and can be adjustable between 10 and 200 nm/min. The pedestal structure is then created by partially removing the underlying silicon dioxide using an isotropic buffered oxide etch (“BOE”). A notch of a narrow oxide strip is formed below the silicon nitride waveguide that supports the upper mid-IR transparent planar structure.

The optical properties, including both the index of refraction “n” and extinction coefficient “k” of the nitride film are characterized by infrared variable angle spectroscopic ellipsometry (“IR-VASE”), a technique that measures and analyzes the polarization change from the reflected mid-IR light. Since the optical constants of nitride films depend on the deposition technique and condition, it is advantageous to know the optical constants to augment the performance of the mid-IR devices.

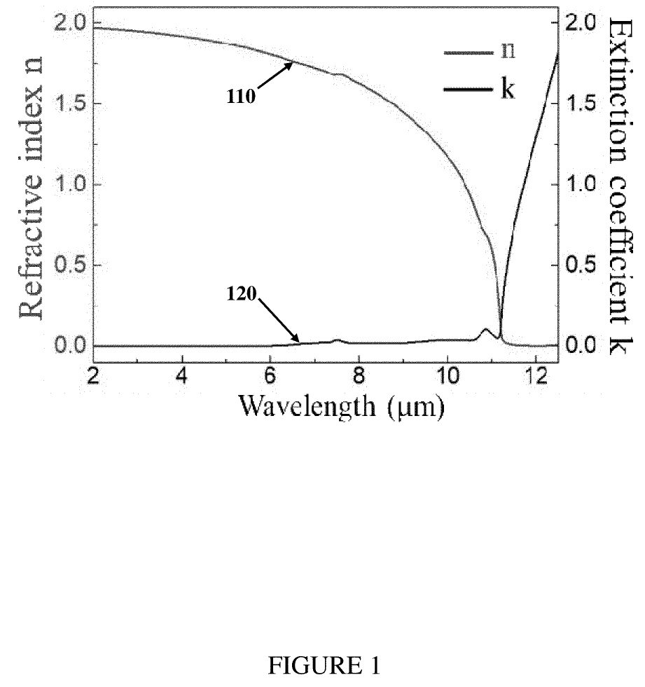

Turning now to FIG. 1, illustrated is a graphical representation of exemplary properties of a nitride film such as an aluminum nitride film. The graphical representation illustrates a refractive index “n” (also designated 110) and extinction coefficient “k” (also designated 120) as a function of wavelength (in micrometers (“μm”)) for the aluminum nitride film. A comprehensive characterization may be accomplished from the near-IR (“NIR”) to the mid-IR. As illustrated in FIG. 1, refractive index 110 decreases slowly from about 2 at a wavelength of 2 μm to about 1.9 at a wavelength of 4 μm before a strong dispersion is found after a wavelength of about 7 μm. The rise of the extinction coefficient 120 is due to the aluminum nitride stretching absorption. Furthermore, absorptions from the nitrogen-hydrogen (“N—H”) stretch at a wavelength of 3 μm.

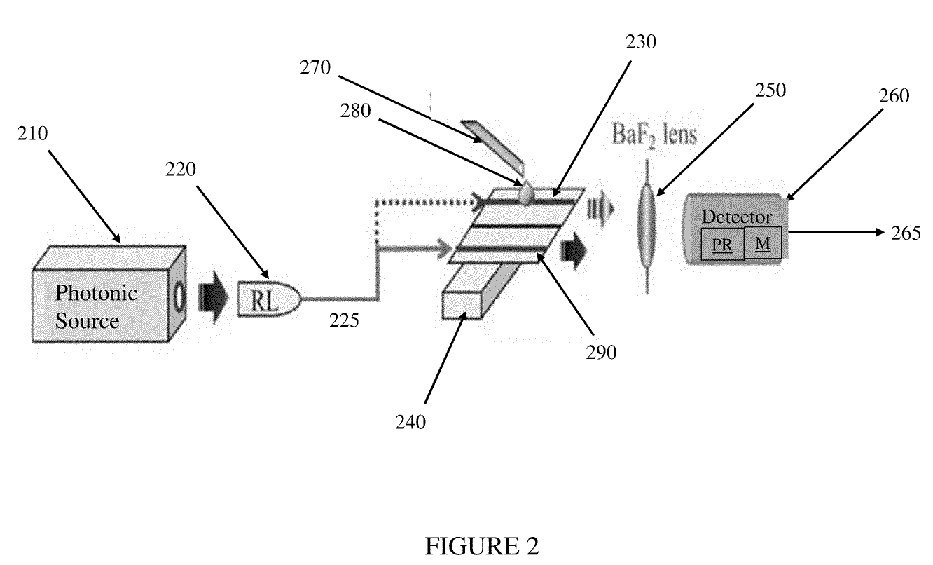

Turning now to FIG. 2, illustrated is a simplified diagram of an embodiment of at least a portion a sensor. A photonic source (e.g., a tunable mid-IR photonic source with tunable wavelength from 2.4 μm to 3.8 μm) 210 is collimated onto an optical fiber (e.g., a mid-IR optical fiber) 225 via a reflective lens (“RL”) 220. Mid-IR signals from on-chip sensors are captured by a detector (e.g., an indium antimonide (“InSb”) mid-IR camera) 260, wherein a barium fluoride (“BaF₂”) lens 250 is placed between waveguides (e.g., mid-IR transparent semiconductor waveguides) 230 and the detector 260 to sharpen the observed waveguide mode. The mid-IR transparent materials include, without limitation, silicon nitride and aluminum nitride (“Al₂N₃”). A volume of 0.5 milliliters (“mL”) of an analyte 280 from a solution such as a glucose solution is dropped from a syringe 270 onto the waveguides 230. The waveguides 230 are supported by a pedestal (e.g., a low refractive index pedestal) 240. A reference waveguide 290 that is not wetted by the glucose solution is utilized as a reference. Signals produced by the detector 260 are processed with a processor (“PR”) coupled to a memory (“M”) therein to produce a signal 265 indicating a glucose concentration present in the analyte 280 in response to an amplitude of a measured photon flux.

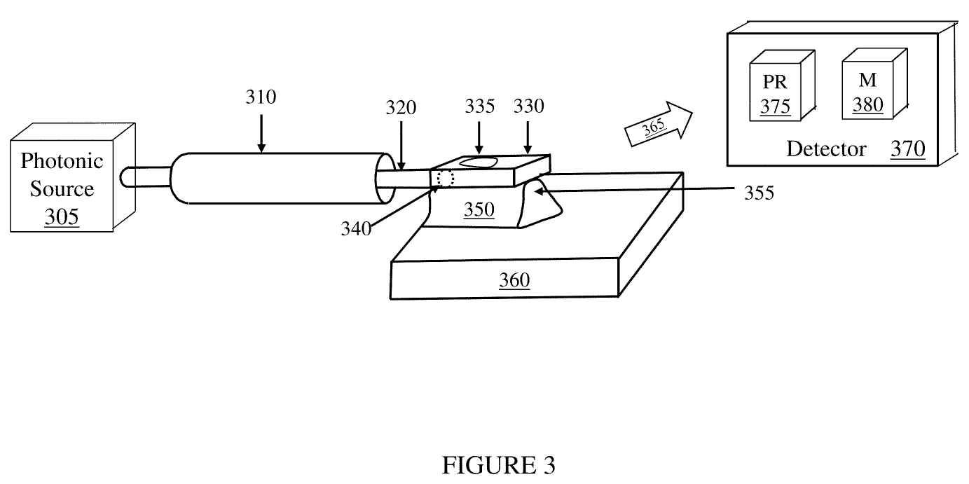

Turning now to FIG. 3, illustrated is a diagram of an embodiment of at least a portion of a sensor. The sensor includes an optical fiber (e.g., a single-mode fluoride fiber) 310 including a fiber core (e.g., a 9 μm fiber core) 320 aligned with a center of a waveguide (e.g., a mid-IR transparent semiconductor waveguide) 330. As shown in FIG. 3, probe light from a photonic source 305 passes through the fiber core 320 coupled to a front facet 340 of the waveguide 330. To improve coupling efficiency, the position of the fiber core 320 is adjusted so as to line up efficiently with the waveguide 330.

During measurements, an analyte 335 such as a glucose solution with various concentrations are prepared based on weight percentages. The waveguide 330 is thereby exposed to the analyte 335 on top and lateral surfaces thereof and at least partially on a lower surface thereof. The absorption strength of the glucose solution is equivalent to the difference of light intensities between the wetted and reference waveguides. To characterize the absorption spectrum of dry analyte molecules, the wetted waveguide 330 is left to dry before measuring the light intensity. The waveguide 330 is supported by a pedestal (e.g., a low refractive index silicon dioxide or aluminum oxide pedestal) 350 including a notch 355 formed on substrate (e.g., a silicon substrate) 360. Mid-IR signals 365 are captured by a detector 370 including a processor (“PR”) 375 and memory (“M”) 380 from the waveguide 330 to produce information about the analyte 335 resident thereon.

The processor 375 may be embodied as any type of processor and associated circuitry configured to perform one or more of the functions described herein. For example, the processor 375 may be embodied as or otherwise include a single or multi-core processor, an application specific integrated circuit, a collection of logic devices, or other circuits. The memory 380 may be embodied as read-only memory devices and/or random access memory devices. For example, the memory 380 may be embodied as or otherwise include dynamic random access memory devices (“DRAM”), synchronous dynamic random access memory devices (“SDRAM”), double-data rate dynamic random access memory devices (“DDR SDRAM”), and/or other volatile or non-volatile memory devices. The memory 380 may have stored therein programs including a plurality of instructions or computer program code for execution by the processor 375 to control particular functions of the apparatus. It should be noted that the processor and memory of FIG. 2 may be analogous to the processor 375 and memory 380 of FIG. 3.

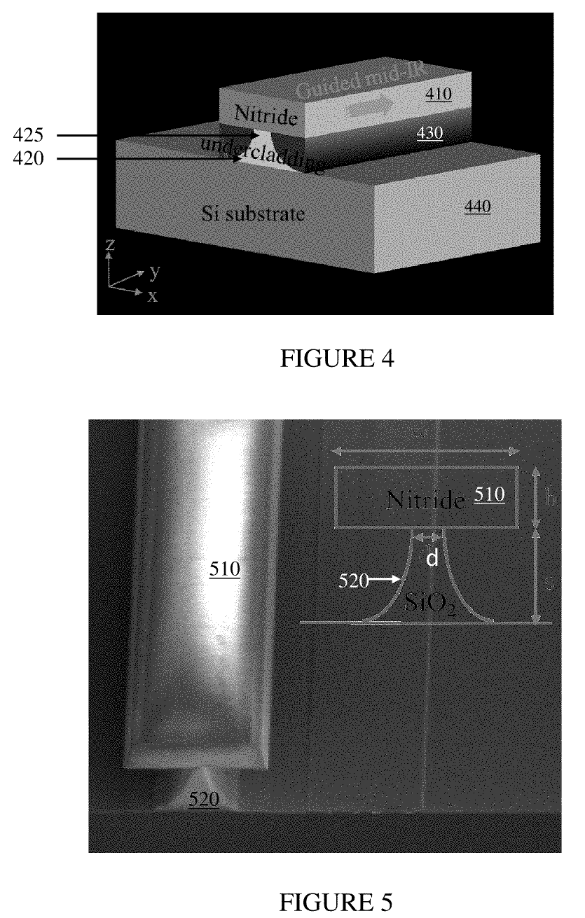

Turning now to FIG. 4, illustrated is a three-dimensional view of an embodiment of at least a portion of a sensor. The sensor (e.g., an on-chip glucose sensor) includes a waveguide (e.g., a mid-IR transparent waveguide 410 such as a mid-IR transparent silicon nitride waveguide) 410 on a pedestal (e.g., an undercladding such as a silicon dioxide pedestal) 420 formed from removing portions of a silicon dioxide layer 430. The pedestal 420 is a low refractive-index undercladding that prevents the nitride guided light from leaking into a substrate (e.g., a high refractive-index silicon substrate) 440. In an embodiment, the waveguide 410 may alternatively be formed with an aluminum nitride. As mentioned above, the pedestal 420 is formed by selectively removing silicon dioxide from the silicon dioxide layer 430 formed on the substrate 440 to form a notch 425 including a narrow strip underneath the waveguide 410 using an isotropic buffered oxide etch. Unlike conventional ridge waveguides that fully attach to the undercladding layer, the bottom surface of the waveguide 410 is extensively exposed to surrounding analyte molecules, which leads to improved sensitivity since the interactive area between the waveguide evanescent field and the surrounding analyte increases. Thus, adjusting the geometry of the waveguide 410 through its pattern designs and etching methods can improve the sensing performance.

Turning now to FIG. 5, illustrated are views of an embodiment of at least a portion of a sensor. A scanning electron microscope view of the sensor includes a waveguide (e.g., a mid-IR transparent waveguide such as a mid-IR transparent silicon nitride waveguide) 510 on a pedestal (e.g., an undercladding such as a silicon dioxide pedestal) 520. A cross-sectional view of the sensor also includes representative dimensional parameters of width (“W”) and height (“h”) of the waveguide 510 and notch width (“d”) and standoff height (“s”) of the pedestal 520. The sensor includes representative dimensions of W=10 μm, h=2 μm, s=5 μm, and d=2 μm. The edges of waveguide 510 may be sharp and the upper and lateral surfaces are smooth, both of which assist in reducing waveguide propagation loss. In addition, the integrated design of the waveguide 510 firmly supported by the pedestal 520 provides a tight coupling to reduce detachments between the waveguide 510 and underlying substrate (see, e.g., FIGS. 3 and 4). This can be proven via a strong breaking stress applied to the sensor applied during wafer cleaving.

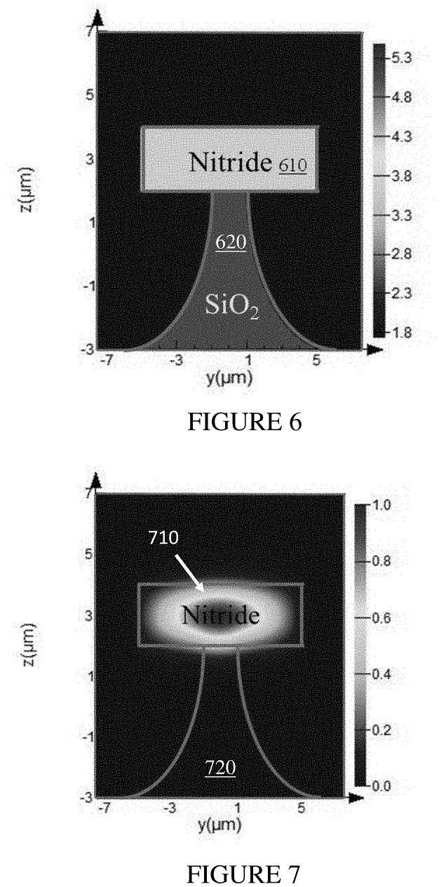

Turning now to FIG. 6, illustrated is a graphical representation showing an exemplary refractive index profile of a portion of a sensor. In the illustrated embodiment, the sensor includes a nitride waveguide (such as a silicon nitride waveguide) 610 having a refractive index of 1.8-2.1 and a silicon dioxide pedestal 620 having a refractive index of 1.5. The mode profiles of the nitride waveguide 610 may be numerically simulated with a method using two-dimensional finite difference method (“FDM”) calculations. The sensor includes representative dimensions of W=10 μm, h=2 μm, s=5 μm, and d=2 μm as set forth above with respect to FIGURES. A light source of 14 μm×10 μm (at x=−7 μm to x=7 μm, y=−5 μm to y=5 μm) was selected so its size would be comparable to a single-mode fiber with a 9 μm core diameter.

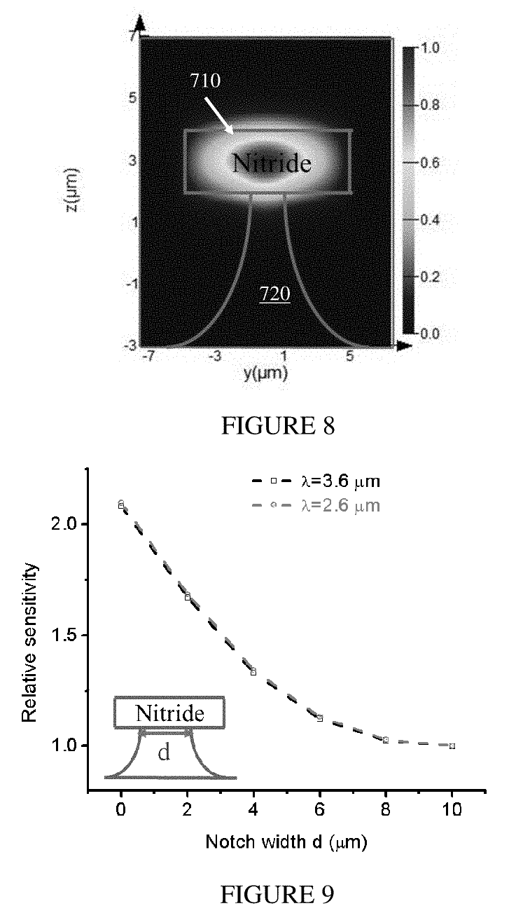

Turning now to FIGS. 7 and 8, illustrated are graphical representations showing exemplary field intensity profiles of a portion of a sensor. The sensor includes a nitride waveguide (such as a silicon nitride waveguide) 710 and a silicon dioxide pedestal 720. The field intensity profiles of air-clad waveguide modes for the nitride waveguide 710 are calculated at wavelengths of 2.6 μm and 3.6 μm for FIGS. 7 and 8, respectively. A fundamental mode is clearly resolved when the light wave is well confined inside the nitride waveguide 710. When the light wavelength increases from a wavelength of 2.6 μm to a wavelength of 3.6 μm, the evanescent fields expand further into height parameter “z” greater than 2 μm and height parameter “z” less than 0 μm. Similar enhancements are also available for the pedestal 720.

Turning now to FIG. 9, illustrated is a graphical representation showing relative sensitivities (absorption sensitivities) as a function of a notch width “d” of a pedestal of a sensor. The relative sensitivities are demonstrated at a wavelength of 2.6 μm (lighter curve) and at a wavelength of 3.6 μm (darker curve). When the notch width “d” decreases from 10 μm to 6 μm, the relative sensitivity increases slowly, but it then rises sharply when the notch width “d” is shortened from 6 μm toward 0 μm. A 70 percent improvement is observed in FIG. 9 when the notch width “d” is reduced from 10 μm to 2 μm. This result is independent of wavelength since the plots calculated at a wavelength of 2.6 μm and a wavelength of 3.6 μm substantially overlap. The raised sensitivity from the pedestal is attributed to the extended interaction between the evanescent fields and the surrounding analyte when the underlying silicon dioxide pedestal is carved out.

The sensor as introduced herein is capable of sensing an analyte such as glucose by correlating a measured spectrum with various characteristic absorption bands belonging to glucose molecules. The spectra are acquired by recording intensities and mode images of light exiting from the waveguide during a wavelength scan.

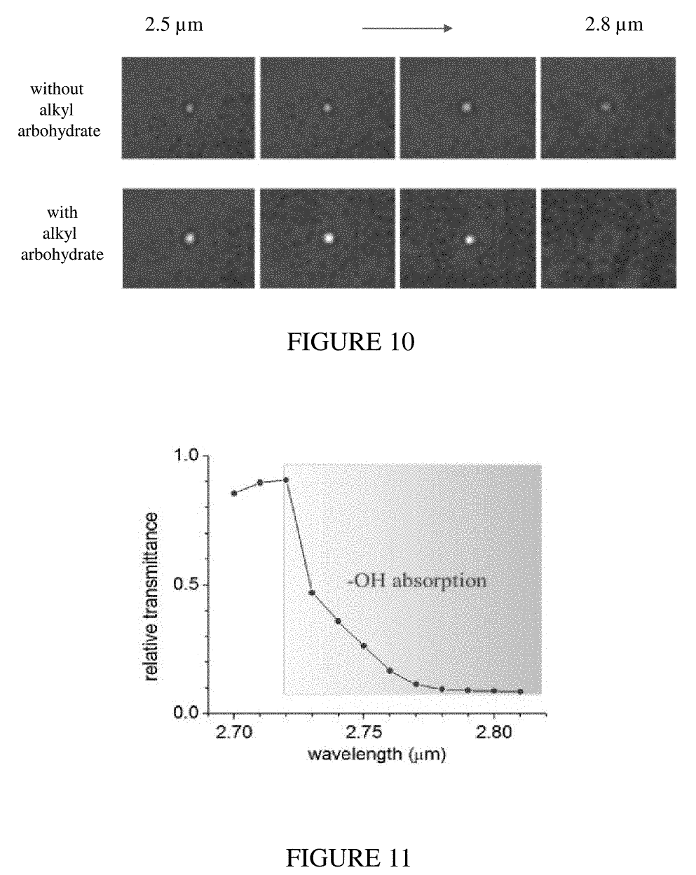

Turning now to FIG. 10, illustrated are mode images of different waveguides without and with alkyl carbohydrate (such as glucose) as the wavelength is gradually tuned from 2.5 μm to 2.81 μm. The waveguide without the alkyl carbohydrate, which may serve as a reference waveguide, shows the same fundamental mode and a constant light intensity throughout the spectral scan. On the other hand, the waveguide with the alkyl carbohydrate demonstrates strong intensity attenuation after the wavelength passes beyond a wavelength of about 2.73 μm.

Turning now to FIG. 11, illustrated is a graphical representation showing relative transmittance of glucose molecules measured by a waveguide. To quantitatively analyze the absorption spectrum, relative transmittance is plotted against wavelength. Here the relative transmittance is defined as (Iᵣ−I_{g})/Iᵣ, where I_{g} and Iᵣ are the measured waveguide intensities covered with and without glucose, respectively. An absorption band rises sharply at a wavelength of 2.73 μm, and then saturates for a wavelength greater than about 2.78 μm. This strong absorption is attributed to the hydroxyl function groups as each glucose molecule has five —OH bonds. By measuring the characteristic —OH absorption, the mid-IR silicon nitride waveguide can achieve a detection limit less than 0.5 nanograms (“ng”) of glucose.

The sensor including the waveguide as described herein is capable of differentiating, without limitation, water and glucose molecules and can also trace a glucose concentration from various aqueous solutions. To evaluate its performance, spectral absorption of water and glucose is characterized using the silicon nitride waveguide at wavelengths of 3.5, 3.6 and 3.7 μm.

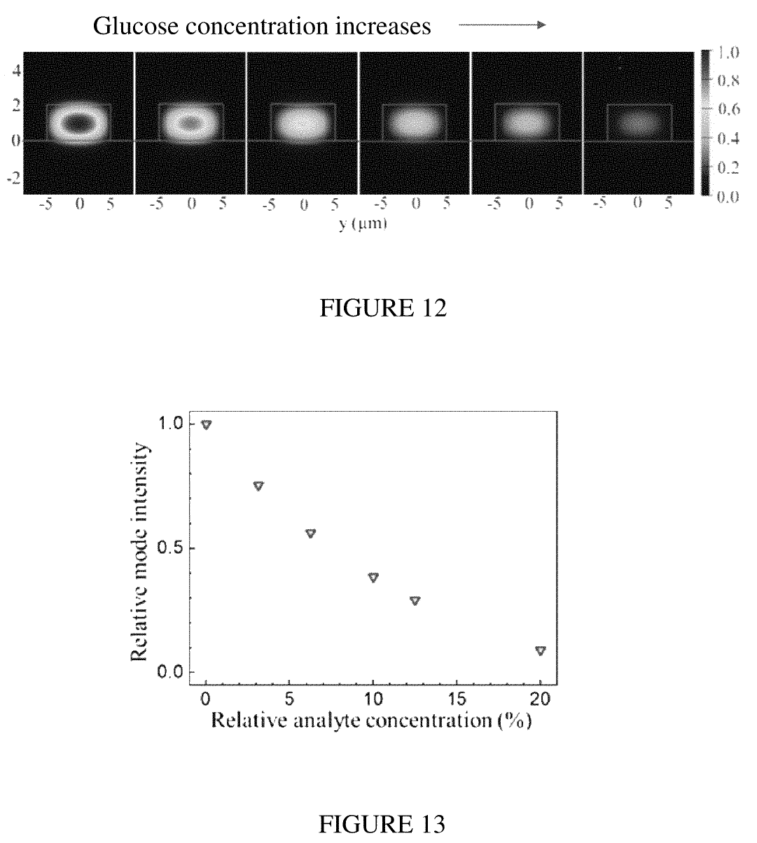

Turning now to FIG. 12, illustrated are mode images of an aqueous glucose concentration at a wavelength of 3.6 μm. The waveguide mode intensity decreases as the glucose concentration increases. For the waveguide with water, the mode intensity sharply increases when the photonic source increases its wavelength from 3.5 μm to 3.6 μm, and an even stronger mode intensity is found at a wavelength of 3.7 μm. The recovery of the mode intensity at a longer wavelength can be explained by the —OH absorption band, which has its absorption edge at a wavelength of 3.55 μm. On the other hand, the mode of the waveguide with the glucose remains dim at a wavelength 3.6 μm, due to the strong absorption by its alkyl group. Thus, by reading the waveguide mode intensities at a wavelength 3.6 μm, one can differentiate glucose from water and trace the glucose concentration from aqueous samples.

Turning now to FIG. 13, illustrated is a graphical representation of relative waveguide mode intensity versus aqueous glucose concentration. To prove the method, relative mode intensity of glucose with relative concentrations in the analyte between 0 and 20 percent are characterized by a sensor at a wavelength of 3.6 μm. The relative mode intensity decreases strongly as the glucose concentration increases in the analyte. These results demonstrate that the sensor as described herein is capable of performing accurate diabetes monitoring.

A widely applicable mid-IR transparent semiconductor waveguide sensor is introduced herein that can provide high sensitivity label-free glucose (or other analyte) detection by guiding mid-IR light with an aluminum nitride, gallium nitride, or silicon nitride waveguide mounted on a silicon or aluminum oxide pedestal at wavelengths from, for instance, 2.70 to 2.81 μm and from 3.50 to 3.70 μm. The sensor can be fabricated using conventional chip-scale technology. Using chip-scale photonic components, a process is enabled to develop a wearable, real-time solution (such as a glucose solution) monitor by leveraging microelectronics fabrication and mid-IR label-free technology. Both of these aspects can be employed for diabetes control, making such a monitor highly desirable for several industry sectors, including public and private healthcare.

To evaluate the sensor for glucose detection, the mid-IR sensor lines up with the characteristic —OH absorption at a wavelength of about 2.80 μm and a sensitivity of less than 0.5 ng. The mid-IR is then shifted to a wavelength of 3.60 μm to measure aqueous glucose concentrations because the alkyl group has a strong absorption at a wavelength of 3.60 μm, whereas the —OH absorption band stops before a wavelength of about 3.55 μm. For instance, a sensitivity of better than 150 milligrams per deciliter (“mg/dL”) can be demonstrated. The high sensitivity is attributed to the long interaction length between the glucose molecules and mid-IR spectral absorption when waveguide geometry is applied. Furthermore, the pedestal structure improves sensitivity by an additional 70 percent since a pedestal waveguide has a larger sensing surface compared to a conventional ridge waveguide. As an example, the refractive index of the pedestal is at least 0.5 less than the refractive index of the mid-IR transparent waveguide.

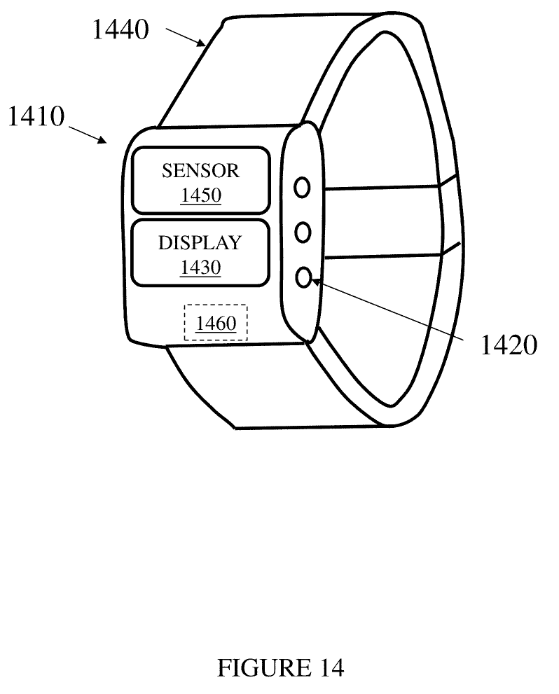

Turning now to FIG. 14, illustrated is a perspective view of an embodiment of a wearable sensor 1410. The wearable sensor 1410 includes control buttons (one of which is designated 1420), a display 1430 and a band 1440 for displaying information about an analyte. The control buttons 1420 and display 1430 provide a human machine interface for the wearable sensor 1410. The band 1440 secures the wearable sensor 1410 to a person's wrist. The wearable sensor 1410 also includes an apparatus (designated “sensor”) 1450 for detecting an analyte as described hereinabove with respect to FIGS. 2 and 3. In the illustrated embodiment, the apparatus 1450 identifies glucose. A power source 1460 such as a battery or solar cell provides power for the apparatus 1450 and, in general, for the wearable sensor 1410.

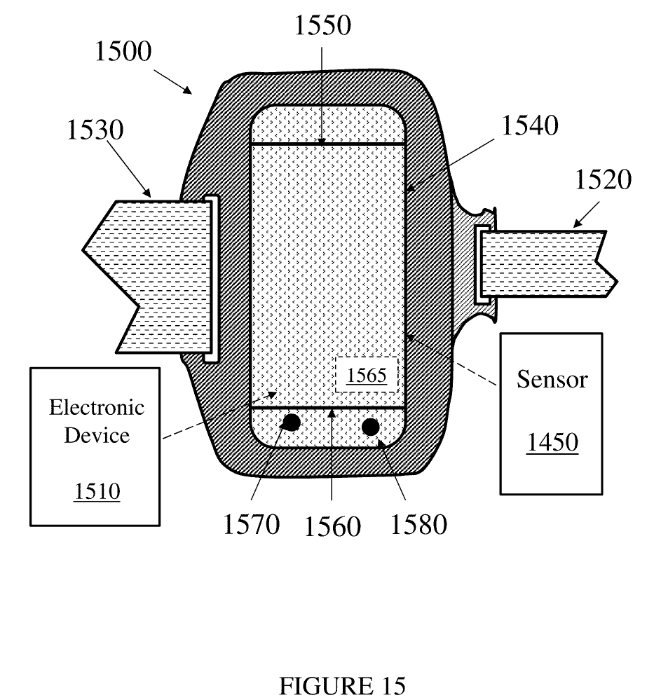

Turning now to FIG. 15, illustrated is a rear view of an embodiment of a retaining device 1500 for the apparatus 1450 for detecting a substance of FIG. 14. The retaining device 1500 includes bands 1520, 1530 operable to be attached to an extremity (e.g., an arm, leg, or wrist) of a person or to an object. The retaining device 1500 includes a cavity 1540 with elastic cords 1550, 1560 that provide a retention mechanism for an electronic device 1510 (e.g., an electronic watch, a multimedia player, a personal fitness sensor, and a medical monitor) and the apparatus 1450. The retaining device 1500 is configured to be worn about an extremity of a person (or to an object) and may provide electrical power via a power source 1565 for the electronic device 1510 and apparatus 1450 that is removably coupled (in this case inserted) into the cavity 1540. The retaining device 1500 also includes electrical contacts 1570, 1580 to provide an electrical connection for the electronic device 1510 and/or the apparatus 1450.

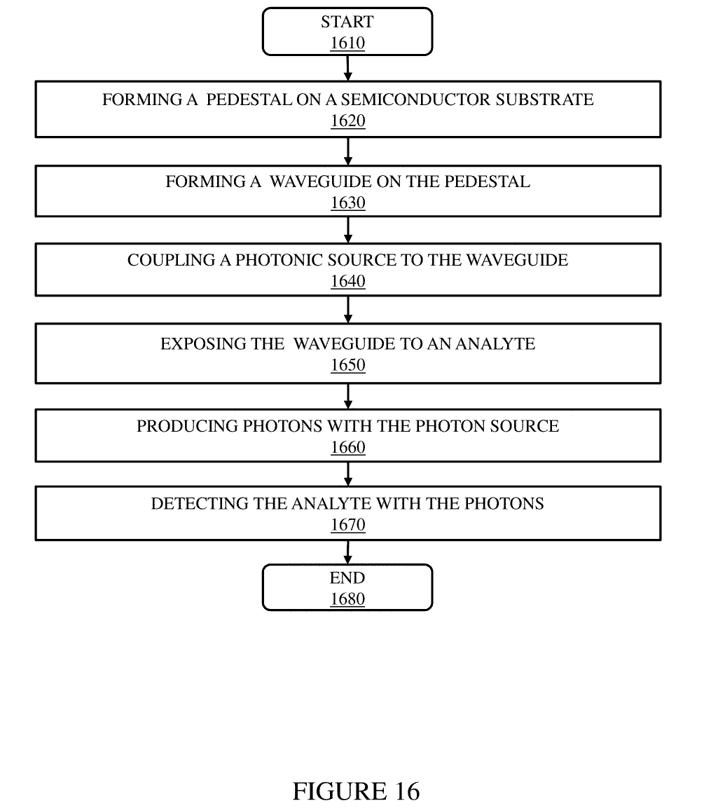

Turning now to FIG. 16, illustrated is a flow diagram of an embodiment of a method for detecting an analyte. The method begins at a start step or module 1610. At a step or module 1620, the method includes forming a pedestal such as a bilayer pedestal on a semiconductor substrate. The pedestal may be a low refractive index pedestal and formed by selectively removing silicon or aluminum oxide from a silicon or aluminum oxide layer formed on the semiconductor substrate to form a notch including a narrow oxide strip to support a waveguide using an isotropic buffered oxide etch.

At a step or module 1630, the method includes forming a waveguide on the pedestal. A refractive index of the pedestal is less than the waveguide. The waveguide may be a mid-IR transparent semiconductor waveguide formed by photolithographically etching a thin film (e.g., a 2 micrometer thin film) with ultraviolet patterning. In an embodiment, the thin film is, without limitation, a silicon or aluminum nitride thin film. In an embodiment, edges of the semiconductor waveguide are formed sharp, and upper and lateral surfaces thereof are formed smooth. As an example, the waveguide is formed with a width of 10 μm and a height of 2 μm.

In an embodiment, the semiconductor substrate comprises silicon, the low refractive index pedestal comprises silicon or aluminum oxide, and the mid-IR transparent semiconductor waveguide comprises a silicon or aluminum nitride waveguide formed on the silicon dioxide pedestal. In an embodiment, a refractive index of the low refractive index pedestal is at least 0.5 less than a refractive index of the mid-IR transparent semiconductor waveguide.

At a step or module 1640, the method includes photonically coupling a photonic source (e.g., a tunable mid-IR photonic source) to the waveguide. In an embodiment, the tunable mid-IR photonic source is coupled to the waveguide with an optical fiber coupled to a front facet of the waveguide. In an embodiment, the tunable mid-IR photonic source is configured to produce photons with a wavelength from 2.4 μm to 3.8 μm.

At a step or module 1650, the method includes exposing the waveguide to an analyte. The analyte may be exposed to top and lateral surfaces of the waveguide, and at least partially on a lower surface thereof to detect the analyte. In an embodiment, the analyte comprises glucose. At a step or module 1660, the method includes producing photons with the tunable mid-IR photonic source directed to the waveguide. At a step or module 1670, the method includes detecting the analyte with the photons produced by the tunable mid-IR photonic source at wavelengths, for instance, from 2.70 to 2.81 μm and from 3.50 to 3.70 μm. In an embodiment, the detecting comprises detecting the analyte with an indium antimonide mid-IR camera. The method ends at a step or module 1680. Processes and devices described herein are not limited to sensing glucose. It is contemplated within the broad scope of the present invention that the sensor can be employed for detecting and measuring other organic, inorganic, and biochemical compounds, and it can be integrated with a microfluidic system.

Thus, an apparatus for detecting an analyte and related methods have been introduced herein. In one embodiment, the apparatus includes a pedestal 350 (e.g., formed with silicon or aluminum oxide) formed on a semiconductor substrate (e.g., formed with silicon) 360, and a mid-IR transparent semiconductor waveguide 330 (e.g., formed with aluminum, gallium or silicon nitride) formed on the pedestal 350. A refractive index of the pedestal 350 is less than (e.g., at least 0.5 less than) the mid-IR transparent semiconductor waveguide 330. Edges of the mid-IR transparent semiconductor waveguide 330 may be sharp and upper and lateral surfaces thereof may be smooth. (See, e.g., appearance of 510 in FIG. 5.) A detector (e.g., an indium antimonide mid-IR camera) 370 of the apparatus is configured to detect an analyte (e.g., glucose) 335 couplable to the mid-IR transparent semiconductor waveguide 330.

The apparatus also includes a tunable mid-IR photonic source 305 photonically coupled to the mid-IR transparent semiconductor waveguide 330. The tunable mid-IR photonic source 305 is coupled to a front facet 340 of the mid-IR transparent semiconductor waveguide 330 via an optical fiber 310. The tunable mid-IR photonic source 305 is configured to produce photons with a wavelength from 2.4 to 3.8 μm. The detector 370 is responsive to photons produced by the tunable mid-IR photonic source 305 at wavelengths, without limitation, from 2.70 to 2.81 μm and from 3.50 to 3.70 μm.

The mid-IR transparent semiconductor waveguide 330 is exposed to the analyte 335 on top and lateral surfaces thereof and at least partially on a lower surface thereof to detect the analyte 335. The pedestal 350 is formed by selectively removing silicon or aluminum oxide from a silicon or aluminum oxide layer 430 formed on the semiconductor substrate 360, 440 to form a notch 355, 425 including a narrow oxide strip underneath the mid-IR transparent semiconductor waveguide 330 using an isotropic buffered oxide etch. As an example, the mid-IR transparent semiconductor waveguide 330 is formed with a width of 10 μm and a height of 2 μm and is formed by photolithographically etching an aluminum or silicon nitride thin film (e.g., a 2 μm thin film) with ultraviolet patterning.

Those skilled in the art should understand that the previously described embodiments of an analyte sensor and related methods of operating and forming the same are submitted for illustrative purposes only. While the analyte sensor has been described in the environment of a detecting glucose, the analyte sensor may also be applied in other environments such as, without limitation, a sensor for other organic or inorganic chemical substances.

As described above, the exemplary embodiment provides both a method and corresponding apparatus consisting of various modules providing functionality for performing the steps of the method. The modules may be implemented as hardware (embodied in one or more chips including an integrated circuit such as an application specific integrated circuit), or may be implemented as software or firmware for execution by a processor. In particular, in the case of firmware or software, the exemplary embodiment can be provided as a computer program product including a computer readable storage medium embodying computer program code (i.e., software or firmware) thereon for execution by the computer processor. The computer readable storage medium may be non-transitory (e.g., magnetic disks; optical disks; read only memory; flash memory devices; phase-change memory) or transitory (e.g., electrical, optical, acoustical or other forms of propagated signals-such as carrier waves, infrared signals, digital signals, etc.). The coupling of a processor and other components is typically through one or more busses or bridges (also termed bus controllers). The storage device and signals carrying digital traffic respectively represent one or more non-transitory or transitory computer readable storage medium. Thus, the storage device of a given electronic device typically stores code and/or data for execution on the set of one or more processors of that electronic device such as a controller.

Also, although the present invention and its advantages have been described in detail, it should be understood that various changes, substitutions and alterations can be made herein without departing from the spirit and scope of the invention as defined by the appended claims. For example, many of the processes discussed above can be implemented in different methodologies and replaced by other processes, or a combination thereof. Also, many of the features, functions, and steps of operating the same may be reordered, omitted, added, etc., and still fall within the broad scope of the various embodiments.

Moreover, the scope of the present application is not intended to be limited to the particular embodiments of the process, machine, manufacture, composition of matter, means, methods, and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An apparatus, comprising:
a pedestal formed on a semiconductor substrate;
a mid-infrared (“IR”) transparent semiconductor waveguide formed on said pedestal, a refractive index of said pedestal being less than said mid-IR transparent semiconductor waveguide; and
a detector configured to detect an analyte couplable to said mid-IR transparent semiconductor waveguide.

2. The apparatus as recited in claim 1 wherein said refractive index of said pedestal is at least 0.5 less than said mid-IR transparent semiconductor waveguide.

3. The apparatus as recited in claim 1 wherein:
said semiconductor substrate comprises silicon;
said pedestal comprises silicon or aluminum oxide; and
said mid-IR transparent semiconductor waveguide comprises aluminum, gallium, or silicon nitride.

4. The apparatus as recited in claim 1 further comprising a tunable mid-IR photonic source photonically coupled to said mid-IR transparent semiconductor waveguide.

5. The apparatus as recited in claim 4 wherein said tunable mid-IR photonic source is coupled to a front facet of said mid-IR transparent semiconductor waveguide via an optical fiber.

6. The apparatus as recited in claim 1 wherein said detector comprises an indium antimonide mid-IR camera.

7. The apparatus as recited in claim 1 wherein edges of said mid-IR transparent semiconductor waveguide are sharp and upper and lateral surfaces thereof are smooth.

8. The apparatus as recited in claim 1 wherein said mid-IR transparent semiconductor waveguide is exposed to said analyte on top and lateral surfaces thereof and at least partially on a lower surface thereof to detect said analyte.

9. The apparatus as recited in claim 1 wherein said pedestal is formed by selectively removing silicon or aluminum oxide from a silicon or aluminum oxide layer formed on said semiconductor substrate to form a notch underneath said mid-IR transparent semiconductor waveguide using an isotropic buffered oxide etch.

10. The apparatus as recited in claim 1 wherein said mid-IR transparent semiconductor waveguide is formed by photolithographically etching an aluminum or silicon nitride thin film with ultraviolet patterning.

11. A method, comprising:
forming a pedestal on a semiconductor substrate;
forming a mid-infrared (“IR”) transparent semiconductor waveguide on said pedestal, a refractive index of said pedestal being less than said mid-IR transparent semiconductor waveguide; and
detecting an analyte couplable to said mid-IR transparent semiconductor waveguide.

12. The method as recited in claim 11 wherein said refractive index of said pedestal is at least 0.5 less than said mid-IR transparent semiconductor waveguide.

13. The method as recited in claim 11 wherein:
said semiconductor substrate comprises silicon;
said pedestal comprises silicon or aluminum oxide; and
said mid-IR transparent semiconductor waveguide comprises aluminum, gallium, or silicon nitride.

14. The method as recited in claim 11 further comprising coupling a tunable mid-IR photonic source to said mid-IR transparent semiconductor waveguide.

15. The method as recited in claim 14 wherein said coupling said tunable mid-IR photonic source comprises coupling said tunable mid-IR photonic source to a front facet of said mid-IR transparent semiconductor waveguide via an optical fiber.

16. The method as recited in claim 11 wherein said detecting said analyte is performed by an indium antimonide mid-IR camera.

17. The method as recited in claim 11 wherein said forming said mid-IR transparent semiconductor waveguide comprises forming sharp edges and smooth upper and lateral surfaces on said mid-IR transparent semiconductor waveguide.

18. The method as recited in claim 11 further comprising exposing top and lateral surfaces and at least partially a lower surface of said mid-IR transparent semiconductor waveguide to said analyte.

19. The method as recited in claim 11 wherein forming said pedestal comprises selectively removing silicon or aluminum oxide from a silicon or aluminum oxide layer formed on said semiconductor substrate to form a notch underneath said mid-IR transparent semiconductor waveguide using an isotropic buffered oxide etch.

20. The method as recited in claim 11 wherein said forming said mid-IR transparent semiconductor waveguide comprises photolithographically etching an aluminum or silicon nitride thin film with ultraviolet patterning.
